(19) United States
(12) Patent Application Publication
(10) Pub. No.: US 2005/0119533 A1
(43) Pub. Date: Jun. 2, 2005

(54) Radiofrequency adapter for medical monitoring equipment

(75) Inventors: Christopher Bryn Sparks, Christchurch (NZ); Geoffrey Kennedy, Christchurch (NZ); Timothy Peter Cussins, Christchurch (NZ); Brett Robert Carpenter, Christchurch (NZ)

Correspondence Address: Chester E. Flavin, Esq.;McCormick, Paulding & Huber LLP, CityPlace II, 185 Asylum Street, Hartford, CT, 06103, US

(73) Assignee: SENSCIO LIMITED, Christchurch (NZ)

(21) Appl. No.: 10/846,222

(22) Filed: May 14, 2004

(30) Foreign Application Priority Data
Nov. 28, 2003 (NZ) 529871

(51) Int. Cl. A61B 5/00

(52) U.S. Cl. 600/300; 128/903; 600/549; 600/323; 600/485; 600/509; 600/529

(57) ABSTRACT

A radiofrequency adapter for medical monitoring equipment which includes a controller which is physically connected to a sensor, and an integrator which is physically connected to a medical monitor; the controller and the integrator are physically separated; the controller provides signal receiving means which receive the signals generated by the sensor, signal conditioning and digitising means, and a radiofrequency transmitter connected to the conditioning and digitising means and arranged to transmit the digitised signals across a wireless radiofrequency link to a radiofrequency receiver incorporated in the integrator; the integrator also includes means for reconstructing the received digital signals back to an analogue signals and means for transmitting the reconstructed and an analogue signals to a monitor physically connected to the integrator.

### TECHNICAL FIELD

The present invention relates to medical monitoring equipment. As used herein, the term “medical monitoring equipment” means equipment designed to receive and analyse signals from sensors connected to or associated with a patient, to monitor one or more medical conditions of that patient. A wide range of medical monitoring equipment is currently available, for monitoring one or more of a number of different conditions. Typically, medical monitoring equipment has the ability to record the analysis of signals from sensors, and often includes an alarm system to alert medical staff of undesirable or dangerous changes in the patient's condition.

### BACKGROUND ART

In the most commonly used types of medical monitoring equipment, a sensor designed to sense the selected condition is attached to the patient and is connected to a monitor by a cable. In use, the sensor generates an analogue signal corresponding to the condition sensed in the patient, and transmits this analogue signal to the monitor via the cable. The monitor then processes/analyses/records readings obtained from the signal. This type of equipment is in widespread use in hospitals throughout the world, and is both reliable and efficient. However, it has the drawback of requiring a cable between the patient being monitored and the monitor. Whilst this is acceptable for short periods of monitoring, it causes numerous difficulties when used over longer periods, since it restricts the scope of the patient's movements. If the patient is asleep or unconscious, the patient may move so as to dislodge the monitor.

To overcome this problem, wireless monitors have been developed, but to date such monitors have not been widely adopted, partly because they are expensive and partly because most hospitals already have a considerable investment in the wired monitors.

### DISCLOSURE OF INVENTION

It is therefore an object of the present invention to provide a radiofrequency adapter for medical monitoring equipment, the adapter permitting the use of known types of sensor and known types of monitor, but without requiring the use of connection cables between the sensor and the monitor. The adapter of the present invention thus permits hospitals to upgrade their existing equipment inexpensively, without any reduction in reliability.

The present invention provides a radiofrequency adapter for medical monitoring equipment which includes: a controller adapted to be physically connected to a sensor; and an integrator adapted to be physically connected to a medical monitor; the controller being physically separated from the integrator;

wherein the controller provides:

a) signal conditioning and digitising means adapted to receive, condition and digitise signals received from the sensor;
b) a radiofrequency transmitter adapted to receive digitised signals from said signal conditioning and digitising means and transmit said signals to said integrator by means of a wireless radiofrequency link;
c) a battery power supply for said signal conditioning and digitising means and radiofrequency transmitter,
   and wherein said integrator provides:
d) a radiofrequency receiver adapted to receive digital radiofrequency transmissions from said radiofrequency transmitter;
e) converting means for converting digital signals received by said receiver to analogue signals;
f) means for transmitting the analogue signals to a monitor physically connected to said integrator.

Preferably, each of the radiofrequency transmitter and the radiofrequency receiver is a radiofrequency transceiver; and each of said signal conditioning and digitising means and said means for converting a digital signal is adapted to convert signals both from analogue to digital and from digital to analogue.

Preferably the integrator further provides sensor control signal sampling means adapted to receive sensor control signals from a monitor connected to the integrator and to transmit said sensor control signals as digitised signals to said controller via said converting means and said radiofrequency transceiver in the integrator; and the controller further provides sensor control signal reconstruction means adapted to receive said sensor control signals from the radiofrequency transceiver in the controller, converted to analogue signals by said signal conditioning and digitising means, and to pass said analogue sensor control signals to the sensor.

The adapter of the present invention may be used in combination with any of a wide range of known medical monitoring equipment and the corresponding sensor, for example:
a) pulse oximetry monitors for measuring the oxygenation levels of the blood.
b) electrocardiograph monitors for measuring cardiac activity.
c) respiration monitors for measuring respiration (e.g. via pressure transducers or airflow or inductance plethysmography or piezoelectric strain gauges).
d) capnography monitors for measuring the carbon dioxide content of breath.
e) blood pressure monitors for measuring blood pressure e.g. using an inflatable cuff.
f) temperature monitors the measuring the body temperature using a thermometer.

The sensors normally used in combination with the monitors listed above are of known type. It will be appreciated that the type of sensor will vary in accordance with the particular characteristic being sensed, but since all of the sensors designed for use with existing medical monitoring equipment operate by producing an analogue signal as described above, any of the sensors may be used in combination with the controller of the present invention, if necessary subject to suitable modification of the controller to receive the physical connection from the sensor and adjustment of the signal conditioning and digitising means as appropriate for the signals from the sensor.

The controller may be mounted in any convenient place on the patient e.g. on one of the patient's limbs, or round the patient's neck, or around the patient's waist.

### BRIEF DESCRIPTION OF THE DRAWINGS

By way of example only, preferred embodiment of the present invention are described in detail, with reference to the accompanying drawings, in which:

FIG. 1 is a block diagram showing the apparatus and signal processing steps of the present invention;

FIG. 2 is a block diagram showing the apparatus and signal processing steps of the present invention as applied to a pulse oximeter monitor; and

FIG. 3 is a diagrammatic exploded view of the adapter of the present invention, as applied to a pulse oximeter monitor.

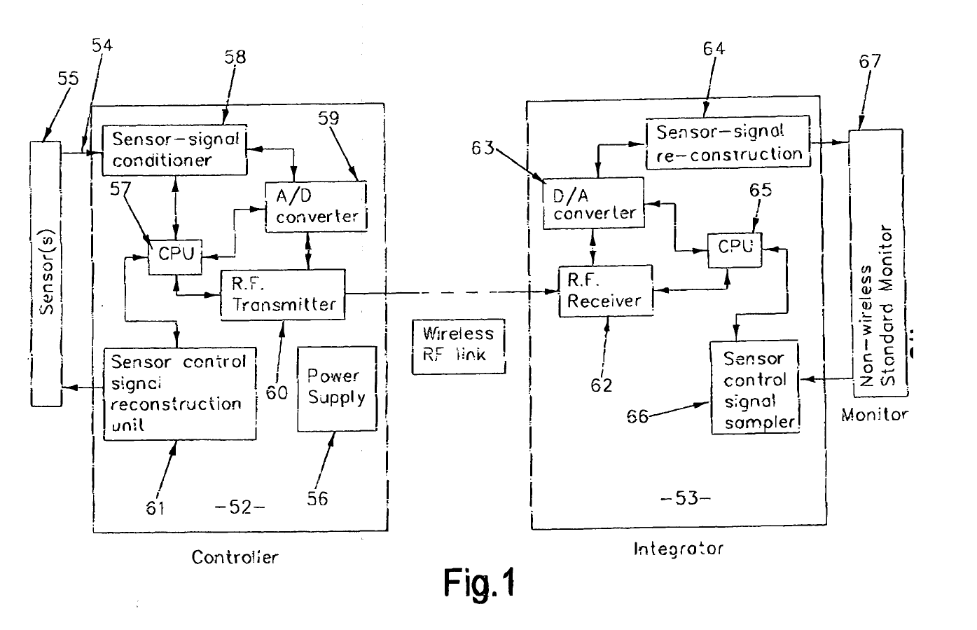

Referring to FIG. 1, an adapter in accordance with the present invention consists of a controller 52 and integrator 53; these two components are designed to be used together but are not physically connected to each other.

The controller 52 is mounted in a housing suitable for attaching to the patient, e.g. by means of a wrist or ankle strap, or by means of a neck strap. The controller is provided with a suitable port 54 through which one or more sensors 55 are physically connected to the controller. The housing of the controller 52 contains a power supply 56 which is electrically connected to a central processing unit (CPU) 57, a sensor signal conditioner 58, an analogue/digital (A/D.) converter 59, a radiofrequency transmitter 60 and (optionally) a sensor control signal reconstruction unit 61. These components are interconnected as shown in FIG. 1, but for clarity the electrical connections between the power supply 56 and the other components have been omitted.

The power supply 56 normally is a battery but, depending upon the intended use of the controller, it may be preferred to provide both a battery and a mains connection, so that the controller 52 can be directly mains powered if necessary. Obviously, using mains power to the controller 52 negates the principal advantage of the invention, and the controller would not normally be used in this manner. However, if the adapter is going to be used for long periods under conditions where accurate monitoring is vital (e.g. during a surgical operation), then the ability to use a mains connection is essential, in case the battery runs flat during the monitoring.

The integrator 53 is adapted to be physically connected to any of a range of known types of medical monitoring equipment, either by mounting the integrator 53 directly on the monitor, or by a cable connection. The integrator 53 may include a battery (not shown) but preferably would be powered by the monitor to which it was attached.

The integrator contains a radiofrequency receiver 82, a digital/analogue (D/A) converter 63, a sensor signal reconstruction unit 84, a central processing unit (CPU) 65, and (optionally) a sensor control signal sampler 66. These components are interconnected as shown in FIG. 1.

The above described equipment operate as follows: the or each sensor 55 is attached to the patient in the appropriate manner, depending upon the nature of the sensor and the condition being sensed. The controller 52 is attached to the patient, and the or each sensor 55 is connected to the controller 52 via the port 54. The integrator 53 is connected to the monitor 67 either directly or via a cable.

The signal from the or each sensor 55 is received by the sensor signal conditioner 58, which manipulates the signal if necessary: typically, the sensor signal conditioner would receive the signal, which would then be amplified and filtered and passed to the A/D, converter 59, which converts the analogue signal to a digital signal. The digital signal is then passed to the radiofrequency transmitter 60, which transmits the digital signal via the wireless RF link to the radiofrequency receiver 62 in the integrator 53. The CPU 57 in the controller 52 may be used to further condition the data contained in the digital signals, and the CPU also may store the data and control the intervals at which the transmitter 60 transmits the data.

The digital signal received by the receiver 82 is passed to the D./A. converter 63 and converted back to an analogue signal. Since the sampling rate of the controller 52 may not correspond exactly to the sampling rate of the monitor 67, the digital signal received by the receiver 62 may be buffered by the CPU 65, by transmitting the digital signal initially to the CPU so that the CPU can output the signal to the D./A. converter 53 at a rate suitable for reception by the monitor 67. The sensor signal reconstruction unit 64 may be used to manipulate the signal for clarity before the signal is passed to the monitor 67. When the signal is received by the monitor 67, it is processed in known manner to get a standard reading as provided by that type of monitor.

Some types of sensor 55 need to be controlled by the monitor 67. This is provided by sending control signals from the monitor 67 to the sensor specific control signal sampler 66 which then generates signals for controlling the sensors in response to instructions from the monitor 67. Such signals ere passed to the CPU 65, digitized by the D./A. converter 63, and are then transmitted to the receiver 62. If control signals of this type are needed, the transmitter 60 and receiver 62 in fact both are transceivers (i.e. transmitter/receivers), so that on receiving a sensor specific control signal, the transceiver 62 can transmit the signal back across the radiofrequency link to the transceiver 60, from which the control signal is passed to the CPU 57, converted to analogue by the A./D. converter 59, passed to the sensor specific control signal reconstruction unit 81 and thus to the sensor 55.

It should be noted that the D./A. and A./D. converters both can convert signals in either direction.

If the sensors 55 do not require control signals from the monitor, then the sensor control signal reconstruction unit 61 and the sensor control sampler 66 are not needed and may be omitted from the controller and integrator respectively.

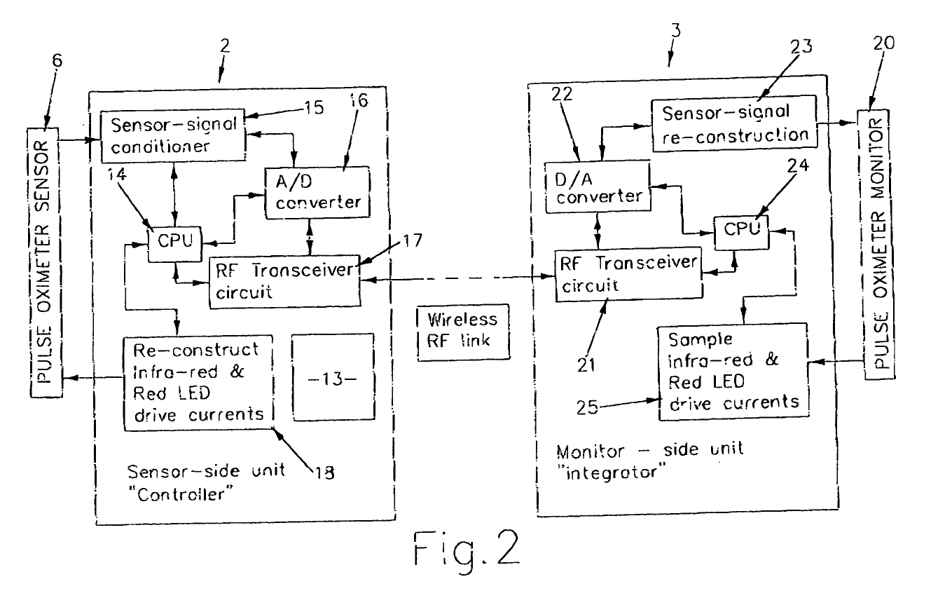

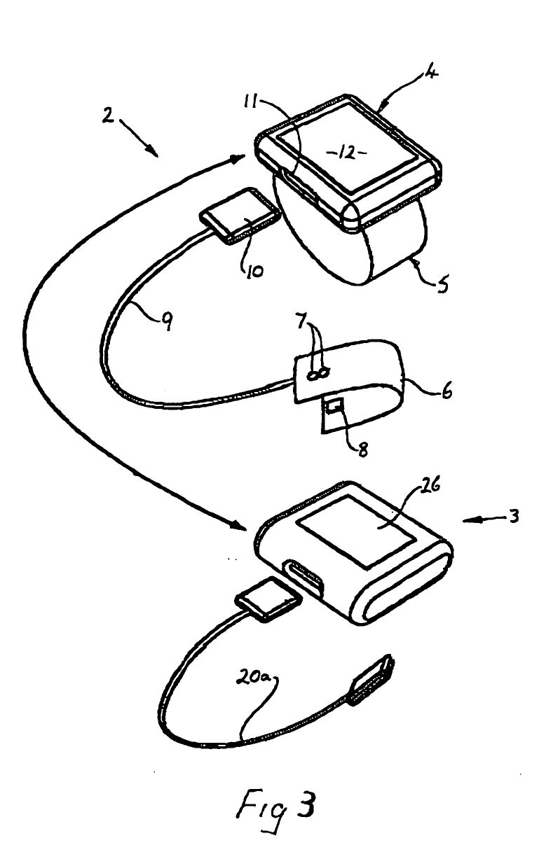

FIGS. 2 and 3 illustrate an embodiment of the invention designed specifically for pulse oximetry.

Oximetry relies on the change in the absorption of electromagnetic energy with change in the percentage of oxygen bound to the haemoglobin molecule in blood. The pulse oximeter functions by comparing the light absorption of fully oxygenated and fully deoxygenated haemoglobin passed through a capillary bed. All currently available conventional pulse oximeters use a combination of two wavelengths, normally 660 nm (red) and 940 nm (near infrared), generated in the sensor by a pair of light emitting diodes. The light is measured with a miniature semiconductor photodetector also in the sensor. The signal in either the red or infrared channels is due to the absorption of some of the energy during its transit from light emitting diode to photodetector. The electronics in a pulse oximeter perform the following functions:
a. Amplification of the photodetector signal
b. Separation of the red and infrared plethysmograph signals
c. Switching and control of current through light emitting diodes
d. Adjustment of the gain of one of the two signals to make them equivalent
e. Separation of the pulsatile (or “arterial”) composition of the signal
f. Analogue to digital conversion of the red and infrared signals
g. Calculation of the red:infrared ratio
h. Calculation of the oxygen saturation (SpO₂)
   i. Oxygen saturation=AR²+BR+C where: R=(AC_{R}/AC_{IR})/(DC_{R}/DC_{IR})
   1. where AC_{R} and DC_{R} are respectively the AC and DC components of the red photodetector signal, AC_{IR} and DC_{IR} are respectively the AC and DC components of the near infrared photodetector signal, and A, B, and C are constants determined by curve fitting against the results of standard blood oxygen measurements.
i. Display:
   i. SpO₂
   i.i. Heart rate
   i.i.i. Signal ‘strength’ or pulse detection
j. Control of alarms
k. Storage of trend of SpO₂ for averaging purposes

Pulse oximetry sensors consist of a transmission sensor, which is designed to be secured over a thin part of the body (generally a finger or toe), such that a portion of the sensor carrying red and infrared light sources lies on one side of the body part, and a portion of the sensor carrying red and infrared light photodetectors lies on the opposite side of the body part. The photo detectors sense light from the red and infrared light sources modulated by passing through the body part and generate a corresponding analogue signal.

Referring to FIG. 3 of the drawings, an adapter in accordance with the present invention consists of a controller 2 and an integrator 3; these two components are designed to be used together but are not physically connected to each other.

The controller 2 consists of a housing 4 which is provided with a securing strap 5 (e.g. a wrist strap) to enable the controller to be attached to the patient, close to the part of the patient's body which is carrying the sensor 6. The sensor 6 is a pulse oximeter sensor of known type, formed as a finger stall with infrared and red light emitting diodes (LEDs) 7 mounted on one side and photo detectors 8 mounted an the other side. The sensor 6 is connected by a shielded cable 9 to a plug 10 which is connectable to a port 11 on the controller 2.

The housing 4 of the controller 2 optionally has a display panel 12 (e.g. an LCD display) on its upper surface. The housing 4 contains a power supply 13 which is electrically connected to a central processing unit (CPU) 14, a sensor-signal conditioner 15, an analogue/digital (A/D) converter 16, a radiofrequency transceiver 17, and an LED driver (reconstruction unit) 18. The components are connected as shown in FIG. 2; for clarity, the electrical connections between the power supply 13 and the other components have been omitted.

The power supply 13 normally would be a battery, but for safety reasons may also incorporate provision for a mains connection, so that the controller 2 can be directly mains powered if necessary.

The integrator 3 is adapted to be physically connected to any of a range of known types of pulse oximeter monitors 20, either by mounting the integrator directly on the monitor or by a cable connection. FIG. 3 depicts connection by a cable 20a. The pulse oximeter monitor 20 is of known type and will not be described in detail; however, it should be noted that the pulse oximeter monitor 20 is of a type which is designed to be physically connected to the pulse oximeter sensor 6.

The integrator 3 may include a battery (not shown) but preferably would be powered by the monitor 20.

The integrator 3 contains a radiofrequency transceiver 21, a digital/analogue (D./A.) converter 22, a sensor signal reconstruction unit 23, a central processing unit (CPU) 24 and an LED drive current sampler 25. The outer housing of the integrator provides a feedback interface 26 (e.g. an LCD display)

The above described equipment operates as follows: the sensor 6 is secured to a patient's finger in the usual manner, and the controller 2 is secured around the patient's wrist using the strap 5. The sensor 6 is connected to the controller by means of the cable 9. The integrator 3 is connected to the pulse oximeter monitor 20 as described above.

The LED driver 18 in the controller is powered by the power source 13 and controlled by the CPU 14 to supply power to the LEDs 7, the power supply being intermittent so that the red and infrared LEDs 7 pulse an end off in known manner. The switching frequency of the LEDs is selected to allow the adapter to reproduce the analogue signal in the integrator 3 in a form and at a strength suitable for processing by the monitor 20.

The photo detectors 8 an the sensor 6 sense the light from the LEDs as modulated by a passing through the patent's finger, as described above, and generate a corresponding analogue signal which passes to the controller 2 by the cable 9. The brightness of both the red and infrared LEDs can be altered by the CPU 14 and driver 18 to optimise the detection of the light by the photo detectors 8.

The signal from the photo detectors 8 is received by the sensor signal conditioner 15, where the signal is manipulated if necessary: typically the signal received from the sensor would be amplified, filtered, and passed to the A./D. converter 16 where the analogue signal is converted to a digital signal. To improve the accuracy of conversion, a known D.C. current may be subtracted from the signal from the sensor; this known current varies dynamically and it adjusts the sample signal to be within a predetermined band of values to give the best accuracy in the digitised signal. The digital signal is received by the radiofrequency transceiver 17 at intervals controlled by the CPU 14, which may further condition the data and may store the data in a buffer to ensure that the data is transmitted at the correct timing. The data is then transmitted to the radiofrequency transceiver 21 in the integrator 3.

The signal transmitted from the transceiver 17 to the transceiver 21 normally would include other components as well: for example, a controller identification signal (in case of more than one controller is being used in a given area) and information on the status of the power supply 13. The number of times per minute that the digital signal is transmitted is selected to achieve an optimum balance between maintaining the data from the sensor 6 up-to-date, managing the use of the radio bandwidth, and economical use of the power from the power supply 13.

It should be noted that multiple controllers can be used within radio range of each other by use of well-known techniques (e.g. narrowband frequency sharing or random time transmissions). Each controller's transmissions are kept separate by the incorporation of the controller identification signal in each transmission.

The digital signal received by the transceiver 21 is passed to the D./A. converter 22 and converted back to an analogue signal. The sampling rate of the controller 2 may not correspond exactly to the sampling rate of the oximeter monitor 20, so the digital signal received by the transceiver 21 may be buffered by the CPU 24, which receives the digital signal and outputs it to the D./A. converter 22 at a sampling rate suitable for reception by the monitor 20. The analogue signal may be further manipulated for clarity by the sensor signal reconstruction unit 23, before being passed to the monitor 20. When the signal is received by the monitor 20, it is processed in known manner to get a standard oximetry monitoring reading.

The signal received by the monitor 20 may include additional information generated by the sensor e.g. calibration resistor values, and light intensity correction factors.

The LED drive current sampler 25 monitors the control current generated by the monitor 20; this feature is necessary only for some designs of monitor.

For the types of monitor which require this, the sampler 25 passes a signal back to the sensors 6 via the CPU 24, converter 22, transceiver 21, transceiver 17, converter 16 and reconstructor 18.

The above described equipment could be modified to allow information to be displayed, for example using a liquid crystal display panel. A further possible modification would be to add further inputs to the controller to allow the reception and processing of signals from other sensors e.g. cardiac monitoring electrodes.

The present invention has been described from the viewpoint of using a separate adapter for each different type of monitor, but it will be appreciated that it would be possible to use a single adapter for two or more different monitors.

1. A radiofrequency adapter for medical monitoring equipment which includes: a controller adapted to be physically connected to a sensor; and an integrator adapted to be physically connected to a medical monitor; the controller being physically separated from the integrator:
wherein the controller provides:
a) signal conditioning end digitising means adapted to receive, condition and digitise signals received from the sensor;
b) a radiofrequency transmitter adapted to receive digitised signals from said signal conditioning and digitising means and transmit said signals to said integrator by means of a wireless radiofrequency link;
c) a battery power supply for said signal conditioning and digitising means and radiofrequency transmitter;
and wherein said integrator provides:
d) a radiofrequency receiver adapted to receive digital radiofrequency transmissions from said radiofrequency transmitter;
e) converting means for converting digital signals received by said receiver to analogue signals;
f) means for transmitting the analogue signals to a monitor physically connected to said integrator.

2. The adapter as claimed in claim 1, wherein each of the radiofrequency transmitter and the radiofrequency receiver is a radiofrequency transceiver; and each of said signal conditioning and digitising means and said means for converting a digital signal is adapted to convert signals both from analogue to digital and from digital to analogue.

3. The adapter as claimed in claim 2 wherein the integrator further provides sensor control signal sampling means adapted to receive sensor control signals from a monitor connected to the integrator and to transmit said sensor control signals as digitised signals to said controller via said converting means and said radiofrequency transceiver in the integrator; and the controller further provides sensor control signal reconstruction means adapted to receive said sensor control signals from the radiofrequency transceiver in the controller, converted to analogue signals by said signal conditioning and digitising means, and to pass said analogue sensor control signals to the sensor.

4. The adapter as claimed in claim 1 or claim 3 wherein each of the controller and the integrator also provide a central processing unit.

5. The adapter as claimed in claim 1 wherein the controller also provides a mains power supply connection.

6. The adapter as claimed in claim 1 wherein the controller is provided with means for securing the controller to a patient.

7. The combination of the adapter as claimed in claim 1 or claim 3, and a medical monitor selected from the group consisting of:
a) pulse oximetry monitor;
b) electrocardiograph monitor;
c) the respiration monitor;
d) capnography monitor;
e) blood-pressure monitor;
f) temperature monitor.
